(19) United States
(12) Patent Application Publication
(10) Pub. No.: US 2020/0360712 A1
(43) Pub. Date: Nov. 19, 2020

(54) PORTABLE PHOTOTHERAPY DEVICE

(71) Applicant: Aura Topping Technology Co., Taipei City (TW)

(72) Inventor: Chen-Jei TAI, Taipei City (TW)

(73) Assignee: Aura Topping Technology Co., Taipei City (TW)

(21) Appl. No.: 16/785,536

(22) Filed: Feb. 7, 2020

(30) Foreign Application Priority Data
May 17, 2019 (TW) 108117136

(51) Int. Cl. A61N 5/06; H05K 1/14

(52) U.S. Cl. CPC A61N 5/0613; H05K 1/147; A61N 2005/0651; A61N 2005/0626; A61N 2005/0647

(57) ABSTRACT

A portable phototherapy device including a head cap, a printed circuit board, a plurality of flexible circuit boards and a plurality of light emitting diodes (LEDs) is provided. The head cap is formed of a flexible material and is suitable to be worn on a head. The printed circuit board is disposed at a center of the head cap, and has a power input end, a plurality of power output ends and a control signal receiving end. The flexible circuit boards are connected to the printed circuit board, and are built in strip form and distributed octagonally from the center of the head cap. The LEDs are disposed on the flexible circuit boards and electrically connected to the power output ends and the control signal receiving end.

This application claims the benefit of Taiwan application Serial No. 108117136, filed May 17, 2019, the subject matter of which is incorporated herein by reference.

### BACKGROUND OF THE INVENTION

### Field of the Invention

The invention relates in general to a phototherapy device, and more particularly to a portable phototherapy device.

### Description of the Related Art

Laser therapy has been used to cure baldness for many years. In around 1970, professor Mester (Budapest, Hungary), after an initial research, found that illuminating a biological system using a low-level laser light with specific wavelength helps to promote the blood circulation of the biological system. Medical effects of the low-level laser therapy have been disclosed in many relevant researches, and are not repeated here. However, the low-level laser therapy equipment is very expensive and heavy, and normally is only available in hospitals. When the heavy low-level laser therapy equipment is worn on a person, the wearer may feel over-pressure and uncomfortable. Thus, the low-level laser therapy equipment is unportable and is inconvenient to use.

Additionally, due to heavy work pressure, poor eating habits and abnormal lifestyle, modern people are susceptible to illnesses such as headache, insomnia, and mental incompetence. Ordinary people resort to medicines or ejections to maintain their health. However, medicines or ejections are always accompanied by side effects or affect people's lifestyle. Over the time, medicines or ejections may cause burden to the body, and the treatment effects may gradually deteriorate over the time.

### SUMMARY OF THE INVENTION

The present invention relates to a portable phototherapy device, which is convenient to use, adopts physio therapy and reduces burden to the body.

According to an embodiment of the present invention, a portable phototherapy device including a head cap, a printed circuit board, a plurality of flexible circuit boards and a plurality of light emitting diodes (LEDs) is provided. The head cap is formed of a flexible material and is suitable to be worn on a head. The printed circuit board is disposed at a center of the head cap, and has a power input end, a plurality of power output ends and a control signal receiving end. The flexible circuit boards are connected to the printed circuit board, and are built in strip form and distributed octagonally from the center of the head cap. The LEDs are disposed on the flexible circuit boards and electrically connected to the plurality of power output ends and the control signal receiving end.

The above and other aspects of the invention will become better understood with regards to the following detailed description of the preferred but non-limiting embodiment(s). The following description is made with reference to the accompanying drawings.

### BRIEF DESCRIPTION OF THE DRAWINGS

FIG. 1 is a schematic diagram of internal circuits of a portable phototherapy device according to an embodiment of the invention.

FIG. 2 is an appearance diagram and an interior perspective of a portable phototherapy device according to an embodiment of the invention.

FIG. 3 is side view of a portable phototherapy device worn on the head of a person according to an embodiment of the invention.

FIG. 4 is back view of a portable phototherapy device worn on the head of a person according to an embodiment of the invention.

FIG. 5 is front view of a portable phototherapy device worn on the head of a person according to an embodiment of the invention.

### DETAILED DESCRIPTION OF THE INVENTION

Detailed descriptions of the invention are disclosed below with a number of embodiments. However, the disclosed embodiments are for explanatory and exemplary purposes only, not for limiting the scope of protection of the invention. Similar/identical designations are used to indicate similar/identical elements. Directional terms such as above, under, left, right, front or back are used in the following embodiments to indicate the directions of the accompanying drawings, not for limiting the present invention.

According to an embodiment of the invention, a portable phototherapy device capable of stimulating hair growth, curing illnesses such as headache, insomnia, mental incompetence, and weak and cold physique, and improving the circulation of terminal nerves. The user, wearing the portable phototherapy device, can drive or work and at the same time receive photo therapy whenever and wherever the user desires. Besides, the portable phototherapy device can be powered by such as a mobile power and the user can control or switch the portable phototherapy device with a control unit (such as a mobile phone or a control panel) to perform photo therapy to the acupoints on the scalp or perform health maintenance and treatment, such as meridian revitalization, to the head.

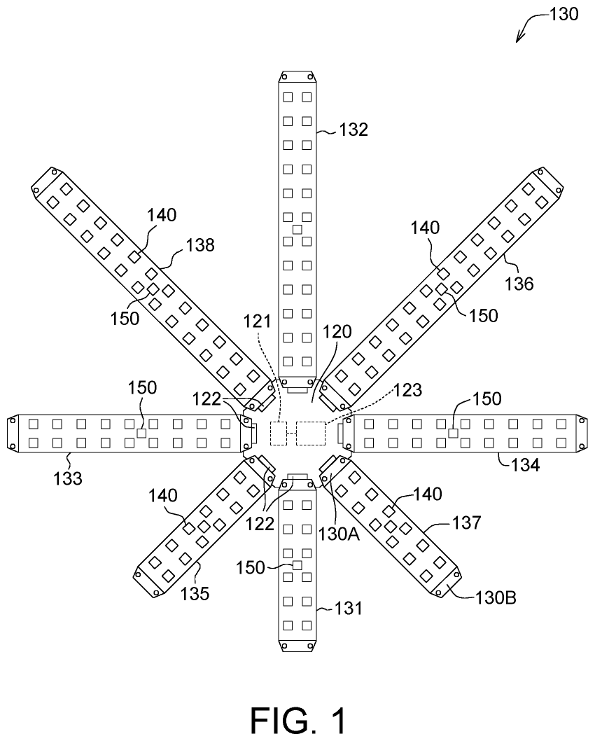

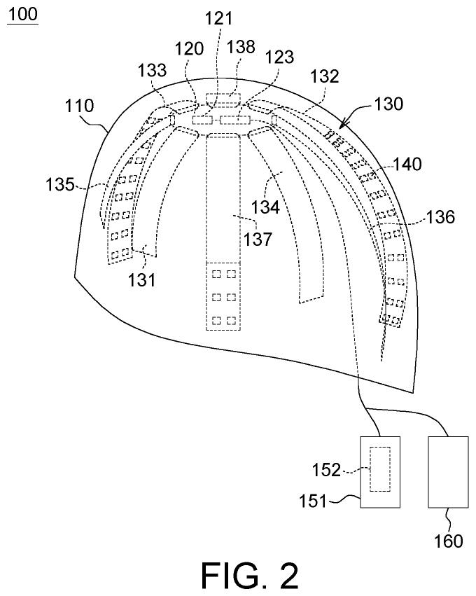

Refer to FIG. 1 and FIG. 2. FIG. 1 is a schematic diagram of internal circuits of a portable phototherapy device 100 according to an embodiment of the invention. FIG. 2 is an appearance diagram and an interior perspective of a portable phototherapy device 100 according to an embodiment of the invention. The portable phototherapy device 100 includes a head cap 110, a printed circuit board 120, a plurality of flexible circuit boards 130 and a plurality of LEDs 140. The printed circuit board 120, the flexible circuit boards 130 and the LEDs 140 are disposed on the inner side of the head cap 110 to provide a necessary power, light source illumination, and the setting of function and photo therapy.

The head cap 110 is formed of a flexible material, such as flannel, nylon cloth, silk cloth, or other cloth formed of cotton, hemp, or yarn. The head cap 110 is suitable to be worn on the hair growing part of the head. The head cap 110 further provides functions such as sun shading, head protection, ventilation and aesthetics. Moreover, the head cap 110 can be formed of an elastic material, which can be flexibly adjusted to fit the shape and size of the user's head, such that the head cap 110 will not easily come off the user's head. When the user wears the head cap 110, the user can adjust the head cap 110 to any position at which the user can receive photo therapy in a comfortable and carefree manner. Besides, the head cap 110, when used in conjunction an outer cap, can be fastened on an inner edge of the outer cap by way of Velcro, fastener, belt or sewing, and looks indifferent from an ordinary cap. Or, the head cap 110 with a lining can be used as a portable energy cap and looks just like an ordinary cap with elements such as the printed circuit board 120 and the flexible circuit board 130 being fixed on the lining by way of Velcro, fastener, belt or sewing.

Refer to FIGS. 1 and 2. The printed circuit board 120 is disposed at a center of the head cap 110 to provide a necessary power to the LEDs 140. The printed circuit board 120 can be realized by a hard insulating substrate, such as an FR-4 glass fiber epoxy substrate whose surface has a plurality of electric terminals. The printed circuit board 120 can be in the shape of an octagon having 8 sides. Besides, the printed circuit board 120 has a power input end 121, a plurality of power output ends 122 and a control signal receiving end 123. The power input end 121 is configured to receive an external power supply 160, such as a mobile battery or an external power supply having a voltage of 5V-12V. The voltage of the power input end 121 can be converted to a voltage of the power output ends 122. Then, the voltage of the power output ends 122 is transmitted to each LED 140 through the flexible circuit boards 130 to enable each LED 140 and make the enabled LEDs 140 illuminate. The power output ends 122 are disposed on each side of the printed circuit board 120 and are electrically connected to the flexible circuit boards 130.

In the present embodiment, the hard insulating substrate is used as the printed circuit board 120. The hard insulating substrate not only provides a necessary power to the LEDs 140 but can further be connected to an external control unit 151, such as a mobile phone or a control panel. A controller or a processor 152 can be disposed in the mobile phone or the control panel to output a control signal. The control signal receiving end 123 of the printed circuit board 120 can receive the control signal through wireless or wired connection to control the light intensity, illumination position, illumination time and parameter setting of each LED 140. For example, the user can use the LEDs 140 to illuminate some or all acupoints on the head, or revitalize one or all meridians of the head or stimulate blood circulation on the said meridians. Therefore, in addition to photo therapy, the invention can further provide a diversity of functions to meet the user's needs.

Refer to FIGS. 1 and 2. The flexible circuit boards 130 are electrically connected between the printed circuit board 120 and each LED 140 to transmit a necessary power and a control signal to the LEDs 140. Each flexible circuit board 130 is formed of flexible wires and a flexible substrate, has the features of lightweight and thinness, can be bent to fit the shape of the head cap 110 and can be fixed on the inner side of the head cap 110, such that the user's head can tightly lean on the head cap 110 and the flexible circuit boards 130 to increase the treatment effect of photo therapy, and the head cap 110 would not be too heavy and make the user feel uncomfortable.

Moreover, the LEDs 140 are disposed on the flexible circuit boards 130 and are electrically connected to the power output ends 122 and the control signal receiving end 123 to provide a light with specific wavelength such as a red light with a wavelength of 650 nm or 660 nm or an infrared light with a wavelength of 850 nm. The LED 140 emits an output energy of 10 W to illuminate the acupoints on the head and provide energy to the hair follicles to stimulate hair growth. Additionally, the portable phototherapy device 100 of the invention can further have at least one temperature sensor 150 disposed on the flexible circuit boards 130. The temperature sensor 150 is configured to detect the temperature on the surrounding of the scalp and the temperature change after the scalp has absorbed the heat, such that the user can receive photo therapy under a comfortable temperature (such as below 40° C.).

Refer to FIG. 1, the two ends of each flexible circuit board 130 respectively have a first fixing plate 130A and a second fixing plate 130B, wherein the first fixing plates 130A are respectively connected to the power output ends 122 of the printed circuit board 120 and the second fixing plates 130B are respectively connected to the head cap 110, such that the flexible circuit boards 130 can be fixed on the inner side of the head cap 110. The first fixing plates 130A and the second fixing plates 130B can be formed of hard insulating substrates, such as FR-4 glass fiber epoxy substrates. The first fixing plates 130A and the second fixing plates 1306 can reinforce the structure of the flexible circuit boards 130 to avoid the two ends of the flexible circuit boards 130 being pulled out and damaging the flexible circuit boards 130. In an embodiment, the first fixing plates 130A can be fixed on the printed circuit board 120 by a conductive adhesive or a solder, and the second fixing plates 130B and a portion of the flexible circuit board 130 can be fixed on the head cap 110 by way of Velcro, fastener, belt or sewing.

Refer to FIGS. 1 and 2, the flexible circuit boards 130 are built in strip form and distributed octagonally from the center of the head cap 110. The flexible circuit boards 130 can also be built in strip form and distributed polygonally from the center of the head cap 110 in the shape of a triangle, a square, a pentagon or a hexagon. The amount of flexible circuit boards 130 is not limited to 8 and can be exemplified by 2, 4, 6, 8 or other number, and the polygonal shape is not limited to “ [symbol] ” as indicated in FIG. 1. For example, when the amount of flexible circuit boards 130 is 2, the two flexible circuit boards 130 can be arranged as “−”; when the amount of flexible circuit boards 130 is 4, the four flexible circuit boards 130 can be arranged as “+”; when the amount of flexible circuit boards 130 is 5, the five flexible circuit boards 130 can be arranged as “★”; when the amount of flexible circuit boards 130 is 6, the six flexible circuit boards 130 can be arranged as “*”. The above arrangements basically are symmetric. However, the flexible circuit boards 130 can also be arranged in an asymmetric manner as long as the illuminated area of the LEDs 140 can cover the acupoints and meridians at the front, the back, the left-hand side and the right-hand side of the head.

Refer to FIG. 1. The flexible circuit boards 130 can be extended to the same or different lengths from the center of the head cap 110, and the amount of LEDs 140 disposed on the flexible circuit boards 130 varies with the lengths of the flexible circuit boards 130. For example, the three flexible circuit boards 130 (131, 135, 137) corresponding to the front of the head are shortest and have a length such as less than or equivalent to 80 mm±10 mm, and have a least amount of LEDs 140 less than or equivalent to 12. The two flexible circuit boards 130 (133, 134) corresponding to the left-hand side and the right-hand side of the head are second shortest and have a length such as less than or equivalent to 150 mm±10 mm, and have a second least amount of LEDs 140 less than or equivalent to 18. The three flexible circuit boards 130 (132, 136, 138) corresponding to the back of the head are longest and have a length such as less than or equivalent to 180 mm±10 mm, and have the largest amount of LEDs 140 less than or equivalent to 24.

As disclosed above, when the flexible circuit boards 130 are extended to different directions from the center of the head cap 110, the lengths of the flexible circuit boards 130 will be different. In the present embodiment, the center of the head cap 110 (that is, the position of the printed circuit board 120) corresponds to the top of Bai Hui (GV20) on the head, therefore the length of each flexible circuit board 130 is determined according to the distance from Bai Hui to the acupoints at the front of the head, the distance from Bai Hui to the acupoints at the left-hand side and the acupoints the right-hand of the head, and the distance from Bai Hui to the acupoints at the back of the head. However, the center of the head cap 110 (that is, the position of the printed circuit board 120) can be on the top of an acupoint (Qian Ding, DU21) anterior to Bai Hui or an acupoint (Hou Ding, GV19) posterior to Bai Hui, and the invention does not have specific restrictions regarding the said arrangement.

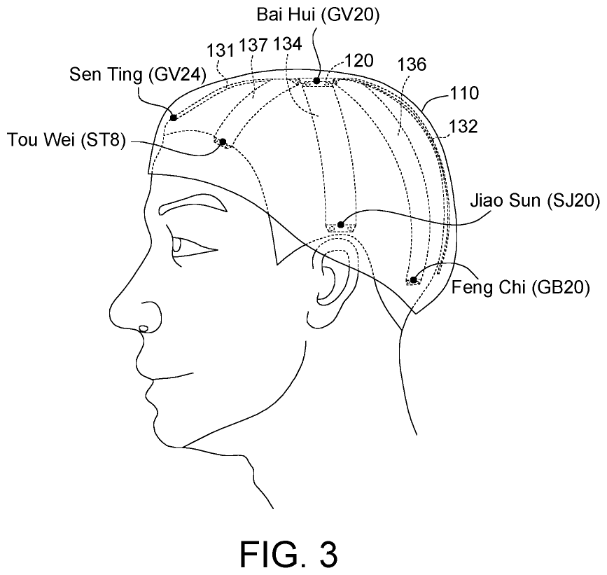

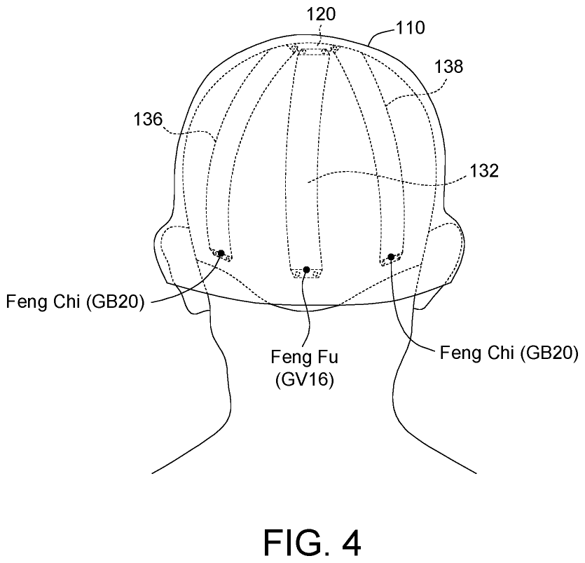

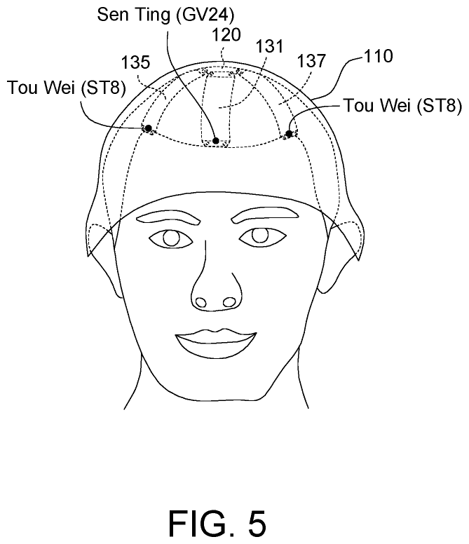

Referring to FIGS. 3-5, a side view, a back view and a front view of a portable phototherapy device 100 worn on the head of a person are respectively shown. Let 8 flexible circuit boards 130 be taken for example. When the portable phototherapy device 100 is worn on the head of a person, the first flexible circuit board 131 is extended to the top of Sen Ting (GV24) (or Shang Xing, GV23) from the top of Bai Hui, and the second flexible circuit board 132 is extended to the top of Feng Fu (GV16) from the top of Bai Hui. The first flexible circuit board 131 and the second flexible circuit board 132 mainly are extended along the Back Middleline (Du Mai) on the head, and pass through the acupoints Sen Ting, Shang Xing, Xin Hui (GV22), Qian Ding (DU21), Bai Hui, Hou Ding (DU19), Qiang Jian (DU18), Nao Hu (DU17), and Feng Fu. Bai Hui is the first acupoint of all yang meridians. The Back Middleline, the taiyang (greater yang) bladder meridian of the foot, the shaoyang (lesser yang) gallbladder meridian of the foot, the yangming stomach meridian of the foot, the taiyang small intestine meridian of the hand, the shaoyang shanjiao (triple heater) meridian of the hand, the yangming stomach meridian of the hand intersect at Bai Hui, which relates to almost all head-related problems. Therefore, performing photo therapy or massage to Bai Hui is very helpful to health maintenance and treatment such as the revitalization of head meridians.

Moreover, Feng Fu, being an acupoint found at the base of the skull, just below the bottom ridge of the skull cap at the top of the neck, is a source for the growth of hair and can further relieve and cure illnesses such as insomnia, neuropathic headache, and dizziness. By performing photo therapy or massage to Feng Fu, health maintenance and treatment such as the revitalization of head meridians can be greatly improved.

Besides, the extending direction of each of the third flexible circuit board 133 and the fourth flexible circuit board 134 is substantially perpendicular to that of the first flexible circuit board 131 and the second flexible circuit board 132. The third flexible circuit board 133 is extended to the top of Jiao Sun (SJ20) at the left-hand side from the top of Bai Hui, and the fourth flexible circuit board 134 is extended to the top of Jiao Sun at the right-hand side from the top of Bai Hui. Jiao Sun is an acupoint located directly above the ear apex and within the hair line. The symptoms of hair loss and gray hairs indicate the weakening of kidney function. The blood cannot be transported to every corner of the scalp, the melanocytes cannot function normally, and hairs turn gray. Therefore, performing photo therapy or massage to Jiao Sun helps to transport nutrition to hair root to stimulate hair growth.

Additionally, the extending direction of each of the fifth flexible circuit board 135 and the sixth flexible circuit board 136 is substantially perpendicular to that of the seventh flexible circuit board 137 and the eighth flexible circuit board 138, and the two directions form an X-shaped cross. The fifth flexible circuit board 135 is extended to the top of Tou Wei (ST8) at the left from the top of Bai Hui; the sixth flexible circuit board 136 is extended to the top of Feng Chi (GB20) at the right from the top of Bai Hui, the seventh flexible circuit board 137 is extended to the top of Tou Wei at the right from the top of Bai Hui; the eighth flexible circuit board 138 is extended to the top of Feng Chi at the left from the top of Bai Hui. Tou Wei, being the intersection point of the yangming stomach meridian of the foot, the shaoyang gallbladder meridian of the foot, and the yangwei meridian, is the transportation and hole of the stomach meridian on the head. By performing photo therapy or massage to Tou Wei, symptoms such as headache caused by pressure or lack of sleep, stomach pain, and insomnia can be relieved. Feng Chi is an acupoint located at the meeting-place of the base of the skull and top of the neck, just lateral to the tendons of the trapezius muscle, and on the shaoyang gallbladder meridian of the foot. By performing photo therapy or massage to Feng Chi, symptoms such as headache, high eye pressure, stiff neck and sore shoulders can be relieved.

As disclosed above, the portable phototherapy device 100 of the invention emits a light with specific wavelength to stimulate acupoints such as Bai Hui, Feng Fu, Feng Chi, and Jiao Sun to promote hair growth. For example, by performing a low-level laser therapy, the LEDs 140 emits a light with a wavelength of 650 nm, 660 nm or 850 nm which penetrates and provides energy to the hair follicle and illuminate the acupoints to stimulate hair growth. The low-level laser therapy can cure the symptoms of hair loss and gray hairs without causing side effect or harming the tissue cells of the body. The user does not need to take medicines. Additionally, the portable phototherapy device 100 of the invention can perform photo therapy to acupoints such as Bai Hui, Feng Fu, Feng Chi, Jiao Sun, and Tou Wei to cure illnesses such as headache, insomnia, mental incompetence, and weak and cold physique, and improve the circulation of terminal nerves without using medicines or ejections and therefore reduce burden to the body.

While the invention has been described by way of example and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A portable phototherapy device, comprising:
a head cap formed of a flexible material and suitable to be worn on a head;
a printed circuit board disposed at a center of the head cap, wherein the printed circuit board has a power input end, a plurality of power output ends and a control signal receiving end;
a plurality of flexible circuit boards connected to the printed circuit board, wherein the flexible circuit boards are built in strip form and distributed octagonally from the center of the head cap; and
a plurality of light emitting diodes (LEDs) disposed on the flexible circuit boards and electrically connected to the power output ends and the control signal receiving end.

2. The portable phototherapy device according to claim 1, wherein the center of the head cap corresponds to the top of Bai Hui (GV20) located at a center of the head.

3. The portable phototherapy device according to claim 1, wherein the flexible circuit boards are outwardly extended to different lengths from the center of the head cap; or, the flexible circuit boards are outwardly extended to different directions and different lengths from the center of the head cap.

4. The portable phototherapy device according to claim 1, further comprising at least one temperature sensor disposed on the flexible circuit boards.

5. The portable phototherapy device according to claim 1, wherein amount of LEDs disposed on the flexible circuit boards varies with lengths of the flexible circuit boards.

6. The portable phototherapy device according to claim 1, wherein two ends of each of the flexible circuit boards respectively have a first fixing plate and a second fixing plate, the first fixing plates are respectively connected to the power output ends, and the second fixing plates are respectively connected to the head cap.

7. The portable phototherapy device according to claim 1, wherein the flexible circuit boards comprise a first flexible circuit board and a second flexible circuit board; the first flexible circuit board is extended to top of Sen Ting (GV24) from top of Bai Hui, the second flexible circuit board is extended to top of Feng Fu (GV16) on the head from top of Bai Hui.

8. The portable phototherapy device according to claim 7, wherein the flexible circuit boards comprise a third flexible circuit board and a fourth flexible circuit board, the third flexible circuit board is extended to the top of Jiao Sun (SJ20) at the left from the top of Bai Hui, and the fourth flexible circuit board is extended to top of Jiao Sun at the right from the top of Bai Hui.

9. The portable phototherapy device according to claim 8, wherein the flexible circuit boards comprise a fifth flexible circuit board and a sixth flexible circuit board, the fifth flexible circuit board is extended to top of Tou Wei (ST8) at the left from the top of Bai Hui, and the sixth flexible circuit board is extended to top of Feng Chi (GB20) at the right from the top of Bai Hui.

10. The portable phototherapy device according to claim 9, wherein the flexible circuit boards comprise a seventh flexible circuit board and a eighth flexible circuit board, the seventh flexible circuit board is extended to top of Tou Wei at the right from the top of Bai Hui, and the eighth flexible circuit board is extended to top of Feng Chi at the left from the top of Bai Hui.
